(19) United States
(12) Patent Application Publication
(10) Pub. No.: US 2024/0166878 A1
(43) Pub. Date: May 23, 2024

(54) FILM-FORMING COMPOSITIONS

(71) Applicant: DDP SPECIALTY ELECTRONIC MATERIALS US 9, LLC, WILMINGTON, DE (US)

(72) Inventors: ANTHONY WENWEI TAN, Midland, MI (US); Gerald K. Schalau, II, Midland, MI (US)

(21) Appl. No.: 18/549,253

(22) PCT Filed: Mar. 30, 2022
(86) PCT No.: PCT/US22/22505

§ 371 Date: Sep. 6, 2023

Related U.S. Application Data
(60) Provisional application No. 63/171,300, filed on Apr. 6, 2021.

(51) Int. Cl. C08L 83/08; A61K 8/898; A61K 9/70; C08G 77/26; C09J 7/38

(52) U.S. Cl. CPC C08L 83/08; A61K 8/898; A61K 9/70; C08G 77/26; C09J 7/38; A61K 2800/54; A61K 2800/805; C09J 2301/302; C09J 2301/408; C09J 2301/502; C09J 2483/00

(57) ABSTRACT

A film-forming composition. comprising: a blend of i) a polysiloxane comprising one or more hydroxyl groups: ii) a polymer comprising a nitrogen-containing repeating unit. wherein the nitrogen-containing repeating unit is an amide. tertiary amine. benzylic amine, or heterocyclic amine: iii) optionally. a solvent: wherein the film formed from the film-forming composition is water-disintegrable.

### CROSS-REFERENCE TO RELATED APPLICATIONS

None

### FIELD OF THE INVENTION

The present invention relates, generally, to film-forming compositions, water-disintegrable films, and methods of making film-forming compositions and films comprising a polysiloxane comprising one or more hydroxyl groups and a polymer comprising a nitrogen-containing repeating unit.

### BACKGROUND OF THE INVENTION

Films and film-forming compositions have healthcare application. For example, they may be used in garments, drapes, wound dressings, bandages, patches and medical devices such as electrodes. Films can be applied to healthy and damaged skin. They can protect the skin and wounds from particulates, bacterial contamination, and abrasion. After application, the films must later be removed from the skin. Therefore, the films must have sufficient adhesion to remain on the skin long enough to achieve a desired result, resist degradation in a particular application, and not cause adverse effects to the patient. For example, the film must resist degradation by biological fluids in wound dressings and be able to be removed from the skin when it's time without causing pain, irritation, or damage to a patient's skin. Not damaging skin is particularly necessary with wounded skin and the skin of elderly patients.

In addition to the adhesive and stability properties, the film or film-forming composition may also need to be a suitable matrix for additives. For example, a suitable matrix may be required for drug actives, antimicrobial, antibacterial, antioxidant, and/or electrically conductive additives.

Organic polymers have had issues in film and film-forming compositions. They provide good adhesive properties but can be difficult to remove from the skin, causing damage, can contain irritating or sensitizing monomers, and have been unsuitable as a matrix for some materials. Polysiloxane materials have also been used in film and film-forming compositions in healthcare applications. Although polysiloxanes offer good removal properties from skin, they have had issues with moisture management, hydrophilicity, and transparency. Combinations of organic and polysiloxane polymers have had incompatibility issues reported.

Therefore, a need exists for film and/or film-forming compositions that are compatible with a range of active material, provide sufficient adhesive strength to remain on the skin as required of the application, but may be easily removed, and that will not adversely affect the skin when in place or removed.

### BRIEF SUMMARY OF THE INVENTION

The present invention is directed to a film-forming composition comprising a blend of i) a polysiloxane comprising one or more hydroxyl groups; ii) a polymer comprising a nitrogen-containing repeating unit, wherein the nitrogen-containing repeating unit is an amide, tertiary amine, benzylic amine, or heterocyclic amine; and iii) optionally, a solvent; wherein the film-forming composition forms a film that is water-disintegrable.

The present invention is further directed to a film comprising i) a polysiloxane comprising one or more hydroxyl groups; and ii) a polymer comprising a nitrogen-containing repeating unit, wherein the nitrogen-containing repeating unit is an amide, tertiary amine, benzylic amine, or heterocyclic amine; wherein the film is water-disintegrable.

The present invention is still further directed to a wearable product comprising the film or film forming composition.

The present invention is still further directed to a method of making a film-forming composition comprising combining a polysiloxane comprising one or more hydroxyl groups, a polymer comprising a nitrogen-containing repeating unit, wherein the nitrogen-containing repeating unit is an amide, tertiary amine, benzylic amine, or heterocyclic amine, and optionally, a solvent, and mixing until a blend is formed.

The film-forming compositions of the invention provide a matrix compatible with a wide range of ingredients and can form films with good adhesive strength that do not irritate, damage, or sensitize skin. The films of the invention are easily removed from the skin without causing damage or irritation to the skin using water.

### BRIEF DESCRIPTION OF THE DRAWINGS

FIG. 1 is a graph of the glass transition temperature (Kelvin) and adhesion force from polycarbonate for blends of polyvinylpyrrolidone K-90 and silanol fluid Dowsil® 4-2737.

FIG. 2 is a graph of heat flow curves obtained from measuring samples shown in FIG. 1 (polyvinylpyrrolidone and Dowsil 4-2737) utilizing differential scanning calorimetry.

FIG. 3 is a graph of glass transition temperature (Kelvin) and adhesion force from polycarbonate for blends of polyvinylpyrrolidone K-90 and silanol fluid Dowsil 4-7042.

FIG. 4 is a graph of glass transition temperature (Kelvin) and adhesion force from polycarbonate for blends of polyvinylcaprolactam (Luviskol® Plus) and silanol fluid Dowsil 4-7042. Solvent was removed from the mixture before measurement. The highest peel force sample corresponds to EXAMPLE 5.

### DETAILED DESCRIPTION OF THE INVENTION

A film-forming composition, comprising:

a blend of
i) a polysiloxane comprising one or more hydroxyl groups;
ii) a polymer comprising a nitrogen-containing repeating unit, wherein the nitrogen containing repeating unit is an amide, tertiary amine, benzylic amine, or heterocyclic amine; and
iii) optionally, a solvent;
wherein a film-forming composition forms films that are water-disintegrable.

A “blend” as used herein is intended to mean that the composition is identical or practically identical wherever it is sampled.

A “homogeneous blend” as used herein is intended to mean a blend that is also indicated by having a single glass transition temperature.

A “water-disintegrable film” as used here in is a film that will change its characteristics by adding water to the film. In some embodiments, the film is a medical adhesive film, and the film will lose, or reduce, its adhesive strength with skin when water is applied to the film.

The polysiloxane i) comprises one or more hydroxyl groups, alternatively from 0.1 to 15% (w/w), alternatively 0.5 to 11% (w/w), based upon the weight of the polysiloxane, of hydroxyl groups. One skilled in the art would know how to determine the weight percent of hydroxyl groups on the polysiloxane.

The hydroxyl groups of the polysiloxane i) may be at the terminus, pendent, or both; or the hydroxyl may be comprised by a group that is terminus, pendant, or both. The hydroxyl may be from a silanol, polyether, hydroxy-substituted hydrocarbyl group, or a combination of a silanol, polyether, and hydroxy-substituted hydrocarbyl group.

The polysiloxane i) may be described as a combination of one or more units represented by the general formula RₐSiO_{(4-a)/2}, wherein “a” has a value from 0 to 3, 1 to 3, or 1.8 to 2.2, and wherein each R is independently hydrogen, hydrocarbyl, hydrocarbyl substituted with one or more hydroxyls, hydroxyl, or a polyether, and wherein at least one R group is hydrocarbyl substituted with one or more hydroxyl groups, hydroxyl, or a polyether.

Hydrocarbyl groups represented by R have from 1-12, alternatively 1-8, alternatively 1 to 6, carbon atoms. Examples of hydrocarbyl groups include, but are not limited to, alkyl such as methyl, ethyl, propyl, 1-methylethyl, butyl, 1-methylpropyl, 2-methylpropyl, 1,1-dimethylethyl, pentyl, 1-methylbutyl, 1-ethylpropyl, 2-methylbutyl, 3-methylbutyl, 1,2-dimethylpropyl, 2,2-dimethylpropyl, hexyl, heptyl, octyl, nonyl, and decyl; cycloalkyl, such as cyclopentyl, cyclohexyl, and methylcyclohexyl; aryl, such as phenyl and naphthyl; alkaryl such as tolyl and xylyl; aralkyl, such as benzyl and phenethyl; alkenyl, such as vinyl, allyl, and propenyl, butenyl, hexenyl, and octenyl; arylalkenyl, such as styryl and cinnamyl; and alkynyl, such as ethynyl and propynyl.

The hydrocarbyl substituted with one or more hydroxyls represented by R is hydrocarbyl as defined above for R except that the hydrocarbyl is substituted by one or more, alternatively 1 or two, alternatively 1, hydroxyls. Examples of the hydrocarbyl substituted with one or more hydroxyl groups include, but are not limited to, hydroxyalkyl, such as hydroxymethyl, 1-hydroxyethyl, 2-hydroxyethyl, 1-hydroxypropyl, 2-hydroxypropyl, 3-hydroxypropyl, 1-hydroxy-1-methylethyl, 2-hydroxy-1-methylethyl, 1-hydroxybutyl, 2-hydroxybutyl, 3-hydroxybutyl, 4-hydroxybutyl, 1-hydroxy-1-methylpropyl, 1-hydroxymethylpropyl, 2-hydroxymethylpropyl, hydroxypentyl, hydroxyhexyl; hydroxycycloalkyl, such as hydroxycyclopentyl, hydroxycyclohexyl, and hydroxymethylcyclohexyl; hydroxyaryl, such as hydroxyphenyl and hydroxynaphthyl; hydroxyalkaryl such as hydroxymethylphenyl and hydroxyxylyl; hydroxyaralkyl, such as hydroxybenzyl and hydroxyphenethyl; hydroxyalkenyl, such as hydroxyvinyl, hydroxyallyl, and hydroxypropenyl; hydroxyarylalkenyl, such as hydroxystyryl and hydroxycinnamyl; alkynyl, such as hydroxyethynly and hydroxypropynyl, and dihydroxyalkly such as 1,2-dihydroxyethyl, 1,2-dihydroxypropyl, and dihydroxyphenyl.

Polyether groups represented by R include, but are not limited to, polyethylene oxide (PEO), polypropylene oxide (PPO), and ethylene oxide and propylene oxide copolymers (PEO/PPO). The degree of polymerization of the PEO, PPO, and PEO/PPO can vary. In one embodiment, the degree of polymerization ranges from 1 to 8, alternatively from 1 to 12, alternatively from 1 to 16.

In one embodiment, the groups represented by R are a combination of methyl and hydroxyl groups, alternatively methyl, phenyl, and hydroxyl groups, alternatively methyl and hydroxy-substituted hydrocarbyl having from 1 to 6 carbon atoms, alternatively methyl and polyether, alternatively methyl, phenyl, and polyether.

The structure of the polysiloxane may vary. For example, the polysiloxane may be, but is not limited to, cyclic, linear, branched, crosslinked, or dendritic.

The polysiloxane may be a mixture of polysiloxanes. In one embodiment, the polysiloxane may be a mixture of polysiloxanes of varying molecular weight, structure, and/or viscosity, alternatively a mixture of polysiloxanes of different structure such as a mixture of linear and cyclic polysiloxanes, alternatively the polysiloxane is linear. In one embodiment, the polysiloxane comprises a substantially linear material, which is end-blocked with a siloxane group of the formula R₃SiO_{1/2}, wherein each R is as defined above.

The degree of polymerization of the siloxane portion of the polysiloxane can vary. In one embodiment, the siloxane portion of the polysiloxane i) has a degree of polymerization from 2 to 200; in another embodiment, from 3 to 150; and in another embodiment, from 3 to 50 silicon atoms.

Examples of polysiloxane i) comprising one or more hydroxyl groups include, but are not limited to, hydroxy-terminated polydimethylsiloxane, poly(methyl vinyl, dimethyl siloxane), poly(methyl phenyl, dimethyl siloxane) and poly(diphenyl, dimethyl siloxane); polyether modified polydimethylsiloxane such as polydimethylsiloxane having terminus or pendant PEO, PPO, or both PEO and PPO, or block copolymers of polydimethylsiloxane and PPO, PEO, or both PPO and PEO; hydroxy-substituted hydrocarbyl terminated or pendant polydimethyl siloxane such as hydroxymethyl-terminated polydimethylsiloxane and hydroxymethyl-terminated copolymers comprising dimethylsiloxane.

One skilled the art would know how to make the polysiloxane. Many polysiloxanes according the invention are available commercially.

The polymer ii) comprises a nitrogen-containing repeating unit, wherein the nitrogen-containing repeating unit is an amide, tertiary amine, benzylic amine, or heterocyclic amine. In one embodiment, the polymer ii) is an organic polymer comprising a nitrogen-containing repeating unit. In one embodiment, polymer ii) is a homopolymer comprising one on or more, alternatively 1 to 3, alternatively 1 or 2, alternatively 1 nitrogen atoms per repeat unit.

The polymer ii) comprising a nitrogen-containing repeat unit can be a homopolymer or have 2 or more, alternatively 2 or 3, different repeat units. When the polymer ii) has different repeating units, at least one of the repeating units comprises a nitrogen.

In one embodiment, the nitrogen atom of the nitrogen-containing repeat unit is resonance stabilized such as with interactions with a mono or polycyclic aromatic carbon, carbonyl groups in an amide, heterocyclic amines, alkynes and vinyl groups.

The polymer ii) has a molecular weight of from 20,000 to 3,000,000 g/mol, alternatively from 100,000 to 2,000,000 g/mol, alternatively from 1,000 to 20,000 g/mol.

The polymer ii) comprising a nitrogen-containing repeating unit forms a composition from a melt or cast of a mixture with the polysiloxane i) comprising one or more hydroxyl groups in a common solvent. In one embodiment, the polymer ii) comprising a nitrogen-containing repeating unit forms a homogeneous composition from a melt or cast of a mixture with the polysiloxane i) comprising one or more hydroxyl groups in a common solvent.

The polymer ii) may comprise two or more polymers comprising a nitrogen-containing repeating unit as described above.

Examples of polymer ii) comprising a nitrogen-containing repeating unit include, but are not limited to polyvinylcaprolatam, polyvinylpiperidone, polyvinylpyrrolidone, polyvinylpyridine, poly(2-ethyl-2-oxazoline), poly(2-methyl-2-oxazoline), poly(N-vinylacetamide), poly(N-isopropylacrylamide), poly(N-vinyl isobutyramide), poly(N-vinyl formamide), poly(N,N-dimethyl acrylamide), polyamino acids, poly(l-vinylpyrrolidone-co-vinyl acetate), polydimethylaminoethyl methacrylate and copolymers thereof. One skilled in the art would know how to make polymer ii). Many polymers comprising an amine in a repeat unit are available commercially.

The solvent is any solvent that will solubilize both polymer i) comprising at least one hydroxyl group, polymer ii) comprising a nitrogen-containing repeat unit, alternatively polymer i), polymer ii) and any additional materials in the film-forming composition. In one embodiment, the solvent is an organic alcohol, alternatively an organic alcohol having from 2 to 10, alternatively 2 to 6, alternatively 2 or three carbon atoms. Examples of solvents include, but are not limited to, organic alcohols such as ethanol and isopropanol. In one embodiment, the solvent is isopropanol; in another embodiment, the solvent is ethanol. In one embodiment, the solvent is an aqueous alcohol solution, alternatively an aqueous alcohol solution comprising 0.1 to 25% (w/w), alternatively 0.1 to 15% (w/w), based on the total weight of the solvent, of water. Many solvents are available commercially.

In one embodiment, the solvent is a mixture of two or more solvents, alternatively a mixture of two or three solvents. Examples of mixtures of solvents include ethanol and isopropanol; ethanol and water; isopropanol and water; and ethanol, isopropanol, and water.

In one embodiment, the mixture in the film-forming composition is a homogeneous blend, wherein “homogeneous blend” has the meaning described above.

The film-forming composition may further comprise additional materials including, but not limited to, a pharmaceutical active, an antimicrobial ingredient, an organic salt, an electrically conductive ingredient, a cosmetic ingredient, and modifiers such as MQ polysiloxane resins. An active is suitable for addition to the film-forming composition if it is soluble or partially soluble in the film-forming composition, alternatively soluble or partially soluble in the polysiloxane having one or more hydroxyl groups. One skilled in the art would know how to determine if a material is soluble or partially soluble in the film-forming composition or the polysiloxane having one or more hydroxy groups. For example, the additive may be mixed into the film forming composition at a typical level

Antimicrobial ingredients, organic salts, and electrically conductive ingredients are known in the art and many are available commercially. Examples of antimicrobial ingredients include, but are not limited to, alkonium halides such as benalkonium chloride and benzyltributylammonium bromide, tetrabutylammonium chloride, and 4-vinylbenzyl(triphenyl)phosphonium chloride.

Cosmetic ingredients are known in the art and are available commercially. Examples of cosmetic ingredients include, but are not limited to, pigments, surfactants, opacifiers, pearlizers, colors, and other classes of ingredients found in the latest edition of the International Cosmetic Ingredient Dictionary.

The film-forming composition comprises from 10 to 85% (w/w), alternatively 30 to 85% (w/w), alternatively 50 to 85% (w/w), alternatively 60 to 85% (w/w), alternatively 30 to 60% (w/w), alternatively 50 to 60% (w/w), based on the weight of the film-forming composition, of polysiloxane i).

The film-forming composition comprises from 15 to 90% (w/w), alternatively from 15 to 70% (w/w), alternatively from 15 to 50% (w/w), alternatively from 15 to 40% (w/w), alternatively from 40 to 70% (w/w), alternatively from 40 to 50% (w/w), based on the weight of the film-forming composition, of polymer ii).

In one embodiment, the film-forming composition comprises solvent, alternatively from 1 to 75% (w/w), alternatively 2 to 20% (w/w), based on the weight of the film-forming composition, of solvent.

When the film-forming composition comprises additional materials as described above, the film-forming composition comprises a sufficient amount of the material to achieve a desired effect, alternatively from 0.01to 10% (w/w), alternatively 0.01 to 5% (w/w) based on the total weight of the film-forming composition, of the additional materials.

The film-forming composition may comprise a pharmaceutical active, alternatively a pharmaceutically sufficient amount, alternatively from 0.01 to 10%, alternatively from 0.01 to 5% (w/w), alternatively 0.01 to 2% (w/w), of a pharmaceutical active. Examples of pharmaceutically active materials include, but are not limited to, lidocaine, camphor, diphenhydramine, terbinafine, clotrimazole, albaconazole, pramiconazole, miconazole, clonidine base. The method of making many pharmaceutical actives are known in the art. Many pharmaceutical actives are available commercially.

The film-forming composition can be used for various applications. The applications in which the film-forming composition may be used include, but are not limited to, biomedical sensing and medical electrodes, adhesives, such as radiation resistant adhesives, antimicrobial films, drug delivery matrix for topic and transdermal drug delivery. One skilled in the art would know how to use the film-forming composition in these various applications.

The film-forming composition may be made by combining with mixing the components in any order until the blend, alternatively the homogeneous blend, is formed. Alternatively, the polysiloxane i) and/or polymer comprising a nitrogen-containing repeating unit ii) are first mixed in a solvent and then combined with mixing until the, alternatively the homogeneous blend, is formed. Any additional materials added to the film-forming composition can be added according to their solubility in either the polysiloxane i), the polymer comprising a nitrogen-containing repeating unit, the solvent, or solvent and either i), ii), or i) and ii). The mixing equipment used to mix the components of the film-forming composition is not critical. One skilled in the art would know what equipment to use and how to combine and mix the composition.

The temperature at which the components of the film-forming composition are combined can vary. For example, the combining may be completed at around ambient temperature, alternatively ambient temperature to elevated temperature, alternatively from 10° C. to 60° C. One skilled in the art would know how to adjust and optimize the temperature at which the components of the film forming composition are combined.

In one embodiment, the film-forming composition is made by mixing the polysiloxane i), polymer ii) and any other ingredients and heating the mixture to near the glass transition temperature of polymer ii) and mixed until homogenization is achieved. In another embodiment a solvent that will solubilize both polysiloxane i) and polymer ii) is added to the polysiloxane and polymer mixture during the homogenization process.

A film, comprising:
i) a polysiloxane comprising one or more hydroxyl groups; and
ii) a polymer comprising a nitrogen-containing repeating unit, wherein the nitrogen-containing repeating unit is an amide, tertiary amine, benzylic amine, or heterocyclic amine;
   wherein the film is water-disintegrable.

The polysiloxane i) and the polymer comprising a nitrogen-containing repeating unit ii) are as described above.

The film may comprise additional materials as described above for the film-forming composition.

The film may be formed by methods known in the art. For example, films may be made by casting the film-forming composition comprising a solvent described above onto a backing sheet and then evaporating the solvent. One skilled in the art would know how to cast films and how to evaporate solvent.

In one embodiment, the film is an adhesive film. The applications in which the film may be used include, but are not limited to, biomedical sensing and medical electrodes, adhesives, such as radiation resistant adhesives, antimicrobial adhesive films, drug delivery matrix with adhesive properties for topic and transdermal drug delivery. Films for these applications may be made by incorporated the corresponding additional materials as described above for the film-forming composition and then casting films from the film-forming composition according to methods known in the art. One skilled in the art would know which additives to incorporate, how to incorporate the additional materials and how to use the films formed from the film-forming compositions for these applications.

A wearable product, comprising: a film-forming composition. The film-forming composition is as described above. One skilled in the art would know how to make and use a wearable product comprising the film forming composition. For example, a film may be cast on a backing and a top sheet; the top sheet may be removed when ready to use, and the exposed film may be applied to the section of the wearable product to be attached to the wearer; once attached, the backing sheet may then be removed, and the wearable product applied to the wearer. Alternatively, the film-forming composition may be applied directly to the wearable product with or without a back or top sheet. Examples of the wearable product comprising the film-forming composition include, but are not limited to, a garment, drape, wound dressing, bandage, patch or medical device

A wearable product, comprising a film formed by a film-forming composition. The film and film-forming composition are as described above. One skilled in the art would know how to make a wearable product comprising the film and the film-forming composition as described above. Standard methods of making a wearable product with the film or film-forming composition may be used. Examples of the wearable product containing a film formed by the film-forming composition described above include, but are not limited to, a garment, drape, wound dressing, bandage, patch or medical device.

A method of making the film-forming composition, comprising:
combining a polysiloxane comprising one or more hydroxyl groups, a polymer comprising a nitrogen-containing repeating unit, wherein the nitrogen-containing repeating unit is an amide, tertiary amine, benzylic amine, or heterocyclic amine, and optionally, the solvent; and mixing until a blend is formed.

The polysiloxane and polymer are as defined above for the film-forming composition.

The combining is conducted using known methods in the industry. One skilled in the art would know how to combine the polysiloxane and polymer.

In one embodiment, the method further comprises combining a solvent with the polysiloxane comprising one or more hydroxyl groups and the polymer comprising a nitrogen-containing repeating unit. The solvent is as described above fore the film-forming composition. One skilled in the art would know how to combine the solvent with the polymer and polysiloxane. For example, the polymer maybe be dissolved in the solvent and then the solvent and polymer solution added to the polysiloxane.

The method of forming a film-forming composition further comprises and additional materials, where the additional materials are as described above for the film-forming composition.

The methods, and compositions of the invention provide film-forming compositions that function as a matrix for wide range of materials, such as pharmaceutical additives, and can form films with good adhesive strength that do not irritate, damage, or sensitize skin. The films formed from the film-forming composition of the invention are easily removed from the skin without causing damage or irritation to the skin using water. Films made from the film-forming composition are more cohesive, adherent and flexible compared to films formed from the polysiloxane i) or polymer ii) by themselves. Applications for the film and film-forming composition include biomedical sensing and medical electrodes, adhesives, such as radiation resistant adhesives, antimicrobial adhesive films, drug delivery matrix with adhesive properties for topic and transdermal drug delivery.

### EXAMPLES

The following examples are presented to better illustrate the method of the present invention but are not to be considered as limiting the invention, which is delineated in the appended claims. Unless otherwise noted, all parts and percentages reported in the examples are by weight. The following Table 1 describes the abbreviations used in the examples:

TABLE 1
List of abbreviations used in the examples.

| Abbreviation | Word |
|---|---|
| g | gram |
| wt | weight |
| % | percent |
| N | Newton |
| hr | Hour |
| ° C. | degrees Celsius |
| NA | Not Applicable |
| μm | micrometer |
| mL | Milliliters |
| Min | Minutes |
| L | Liter |
| cm | Centimeter |

### Methods used in the Examples

### Preparation of Film-Forming Compositions

Solutions of the film-forming agents were prepared by mixing the polysiloxanes and polymers comprising amine group repeating units with isopropyl alcohol (50% by weight) until a homogenous formulation was achieved. Some polymers such as Luviskol Plus, Gaffix VC-713 and PVP/VA E-735 were supplied in solution so no additional solvent was added. Tables 2 and 3 contain details regarding the fluids and polymers which were used in the examples. Table 4 contains the compositions of the film-forming compositions.

### Formation of Films

Solutions of the film-forming agents were coated onto 50 μm thick polyester (Mylar) sheets utilizing an automated Meyer draw-down bar and stainless-steel shims. Shim thickness was varied in order to obtain films with a thickness ranging from 25-50 μm. The coated sheets were then heat treated in an oven at 130° C.for 4 min to remove the solvent. The coated Mylar was then sliced into 25 cm wide strips. Evaluation of the strips was carried out within 24 hours of coating.

### Water Disintegrability

Coated strips were dipped into a 1 L jar of room temperature water 60 times over the course of 1 minute. The coatings were then visually examined for changes in appearance and morphology. The coated strips were placed onto a glass plate with the coating facing upward and a finger was run along the length of the strip. The film was deemed water disintegrable if the surface exhibited lubricity or changes in the coating uniformity (clarity, cohesion, surface features, etc.) were observed visually. Insoluble films were those in which the surface remained intact, transparent and no changes in morphology were observed.

### Film Cohesion and Tack

Coated strips were placed on top of a glass slide with the coating facing upwards. The strip was held down such that a 25 cm gap was present on the surface lengthwise. Cohesiveness and tack were evaluated by contacting the surface of a strip within the exposed 25 cm gap using the forefinger and removing it within a span of 1 second. Films were deemed to be cohesive if little or no residue was transferred onto the forefinger. Non-cohesive films were those in which the film lost its integrity and a significant portion was transferred onto the skin. Tack values were assigned based upon perceived tackiness: 1 (tack-free), 2 (slight tack), 3 (moderate tack), 4 (good tack) and 5 (excellent tack). Films which cohesively failed were given values of 5.

Release from Skin

Strips of the coated film were placed onto the forearm, coating side towards the skin. The films were allowed to dwell on the skin for 5 minutes. Afterwards, the films were peeled by grasping one end of the film and pulling at an 180° angel until the entire film was removed in a span of 2 seconds. The gentleness of the release from human skin was evaluated by assigning a value: 0 (noncohesive, residue remains), 1 (aggressive), 2 (moderately aggressive), 3 (acceptable), 4 (good), 5 (excellent).

### Flexibility

Strips of the coated film were folded in half five times. Films which delaminated, cracked or exhibited signs of mechanical failure were deemed to have failed the test. Films which retained their structural properties were judged to be flexible.

### Homogeneity

Strips of the coated film were observed visually. Optically transparent films were deemed homogeneous. Cohesive films with opacity or haziness were deemed miscible.

### Glass Transition Temperature Evaluation

The glass transition temperature of polymer-silicone blends was measured utilizing a TA 2500 differential scanning calorimeter fitted with a liquid nitrogen cooler.

Samples were created by placing 4-14 mg of the blend into Tzero aluminum sample pans. Samples were annealed for five minutes above the glass transition temperature to remove thermal history and then cooled at 10° C./min to -180° C. The glass transition temperature was determined using the half-height heat capacity method applied to the heat flow curve obtained upon heating at 10° C./min. The results were compared against the glass transition temperature predicted by the Fox equation (1):

$$\frac{1}{{T}_{g , blend}} = \frac{{W}_{polymer}}{{T}_{g , polymer}} + \frac{{W}_{silicone}}{{T}_{g , silicone}} \qquad ( 1 )$$

Where T_{g} is the glass transition temperature of the blend, neat polymer and neat silicone in Kelvin and w is the mass fraction of the polymer and silicone components in the blend. The Fox equation is an established method of predicting the glass transition temperature of homogeneous polymer blends. A homogeneous blend will exhibit a single glass transition and a similar glass transition temperature to that predicted by the equation. Heterogeneous or incompatible blends will exhibit large deviations from the glass transition temperature predicted by the Fox equation and/or exhibit separate transitions for each of the components. A person skilled in the art will be familiar with the method of measuring the glass transition temperature experimentally and determining the glass transition temperature via the Fox equation.

### Materials Used in Examples

The following Table 2 includes the organopolysiloxane and other hydroxyl containing materials used in the examples along with their hydroxyl content.

TABLE 2
Organopolysiloxane and other hydroxyl-containing Materials
used in the Examples with their hydroxyl content.

| Product | Source | Fluid Name | OH wt. % |
|---|---|---|---|
| Dowsil ® 4-7041 | Dow Chemical | PDMS, OH-terminated | 10.1% |
| Dowsil 4-2737 | Dow Chemical | PDMS, OH-terminated | 4.0% |
| DMS-S14 | Gelest | PDMS, OH-terminated | 3.1% |
| DMS-C16 | Gelest | PDMS, Carbinol-terminated | 4.7% |
| Silmer OH Di-10 | SilTech | PDMS, Carbinol-terminated | 3.4% |
| Dowsil 4-7042 | Dow Chemical | Poly(methyl vinyl, dimethyl | 8.3% |
| | | siloxane), OH-terminated | |
| Silmer OHT Di-10 | SilTech | PDMS, Tetra carbinol-terminated | 4.6% |
| Carbowax ® PEG 400 | Dow Chemical | Polyethylene glycol 400 | 8.5% |
| Polyglycol P425 | Dow Chemical | Propane-1,2-diol, propoxylated | 8.0% |
| Terathane ® 650 | Lycra Company | Polytetramethylene Glycol | 2.5% |
| Biowax ® 754 Liquid | Biosiltech | Dimethicone PEG-8 | 1.5-2.9% |
| IPA ACS 99.5% | Fisher Scientific | Isopropyl Alcohol | N/A |

The following Table 3 contains the Polymers with amine-containing repeat units used in the examples.

TABLE 3
Polymers comprising repeat units comprising
amine used in the Examples.

| Product | Source | Polymer Name |
|---|---|---|
| Luviskol ® Plus | BASF | Poly(vinylcaprolactam) |
| Kollidon ® K-90 | BASF | Poly(vinylpyrrolidone) |
| Aquazol ® 500 | Polymer Chemistry | Poly(2-ethyl-2-oxazoline |
| | Innovations | |
| P4VP 100 kg/mol | Sigma Aldrich | Poly(4-vinylpyridine) |
| Gaffix ® VC-713 | Ashland Specialty | Vinyl Caprolactam/Vinyl |
| | Chemicals | Pyrrolidone/Dimethylaminoethyl |
| | | Methacrylate Copolymer |
| PVP/VA E-735 | Ashland Specialty | Poly(1-vinylpyrrolidone-co- |
| | Chemicals | Vinyl Acetate) |

### Examples 1-23; Comparative Examples 1-10

Examples 1-23 and Comparative Examples 1-10 illustrate the properties of film-forming agents which embody the invention (Table 4). Examples 6 and 7 demonstrate that the properties of the invention are comparable when formulated from a solution and from a melt. Comparative examples 1-4 are formulations based upon the prior art which utilize a comparable fluid loading as the invention; the comparative examples are not cohesive whereas those in the invention yield a cohesive film. Comparative examples 5-9 are prior art formulations. The comparative examples yield cohesive films which are aggressive on the skin whereas the invention yields films which are skin-friendly and atraumatic. Comparative examples 9 and 10 compare the performance of commercially available silicone and acrylic adhesives with the invention. The silicone adhesive in comparative example 9 exhibits excellent release and tack properties, however, it is not water disintegrable. The acrylic adhesive in comparative example 10 is aggressive on the skin and is not water disintegrable.

TABLE 4
Film formulations and their properties

| Example | Polysiloxane wt. % | | Polymer wt. % | | Release | Tack | Cohesive | Disintegrable |
|---|---|---|---|---|---|---|---|---|
| 1 | Dowsil ® | 62% | Luviskol ® | 38% | 5 | 4 | Yes | Yes |
| | 4-7041 | | Plus | | | | | |
| 2 | Dowsil | 65% | Kollidon ® | 35% | 5 | 4 | Yes | Yes |
| | 4-7041 | | K90 | | | | | |
| 3 | Dowsil | 58% | Aquazol ® | 42% | 5 | 4 | Yes | Yes |
| | 4-7041 | | 500 | | | | | |
| 4 | Dowsil | 65% | P4VP | 35% | 4.5 | 3 | Yes | Yes |
| | 4-7041 | | | | | | | |
| 5 | Dowsil | 62% | Luviskol | 38% | 5 | 4 | Yes | Yes |
| | 4-7042 | | Plus | | | | | |
| 6 | Dowsil | 65% | Kollidon | 35% | 5 | 4 | Yes | Yes |
| | 4-7042 | | K90 | | | | | |
| 7* | Dowsil | 65% | Kollidon | 35% | 5 | 4 | Yes | Yes |
| | 4-7042 | | K90 | | | | | |
| 8 | Dowsil | 58% | Aquazol | 42% | 5 | 4 | Yes | Yes |
| | 4-7042 | | 500 | | | | | |
| 9 | Dowsil | 65% | P4VP | 35% | 4.5 | 4 | Yes | Yes |
| | 4-7042 | | | | | | | |
| 10 | Dowsil | 62% | Luviskol | 38% | 5 | 4.5 | Yes | Yes |
| | 4-2737 | | Plus | | | | | |
| 11 | Dowsil | 68% | Kollidon | 32% | 5 | 2 | Yes | Yes |
| | 4-2737 | | K90 | | | | | |
| 12 | DMS-S14 | 65% | Luviskol | 35% | 4 | 4 | Yes | Yes |
| | | | Plus | | | | | |
| 13 | DMS-S14 | 67% | Kollidon | 33% | 5 | 3 | Yes | Yes |
| | | | K90 | | | | | |
| 14 | DMS-C16 | 62% | Luviskol | 38% | 4 | 4 | Yes | Yes |
| | | | Plus | | | | | |
| 15 | DMS-C16 | 80% | Kollidon | 20% | 5 | 1 | Yes | Yes |
| | | | K90 | | | | | |
| 16 | Silmer ® OH | 62% | Luviskol | 38% | 3 | 4 | Yes | Yes |
| | Di-10 | | Plus | | | | | |
| 17 | Silmer OH | 75% | Kollidon | 25% | 5 | 1 | Yes | Yes |
| | Di-10 | | K90 | | | | | |
| 18 | Silmer OHT | 68% | Luviskol | 32% | 4 | 4 | Yes | Yes |
| | Di-10 | | Plus | | | | | |
| 19 | Silmer OHT | 70% | Kollidon | 30% | 3 | 5 | Yes | Yes |
| | Di-10 | | K90 | | | | | |
| 20 | Silmer OHT | 70% | Aquazol | 30% | 4 | 2 | Yes | Yes |
| | Di-10 | | 500 | | | | | |
| 21 | Dimethicone | 50% | Luviskol | 50% | 3 | 4 | Yes | Yes |
| | PEG-8 | | Plus | | | | | |
| 22 | Dimethicone | 50% | Kollidon | 50% | 5 | 3 | Yes | Yes |
| | PEG-8 | | K90 | | | | | |
| 23 | Dimethicone | 58% | Aquazol | 42% | 5 | 1 | Yes | Yes |
| | PEG-8 | | 500 | | | | | |
| Comp. 1 | PEG 400 | 65% | Kollidon | 35% | 0 | 5 | No | Yes |
| | | | K90 | | | | | |
| Comp. 2 | PEG 400 | 65% | Luviskol | 35% | 0 | 5 | No | Yes |
| | | | Plus | | | | | |
| Comp. 3 | Polyglycol | 65% | Luviskol | 35% | 0 | 5 | No | Yes |
| | P425 | | Plus | | | | | |
| Comp. 4 | Terathane ® | 65% | Luviskol | 35% | 0 | 5 | No | Yes |
| | 650 | | Plus | | | | | |
| Comp. 5 | PEG 400 | 36% | Kollidon | 64% | 5 | 1 | Yes | Yes |
| | | | K90 | | | | | |
| Comp. 6 | PEG 400 | 36% | Luviskol | 64% | 5 | 1 | Yes | Yes |
| | | | Plus | | | | | |
| Comp. 7 | Polyglycol | 50% | Luviskol | 50% | 2 | 5 | Yes | Yes |
| | P425 | | Plus | | | | | |
| Comp. 8 | Terathane | 50% | Luviskol | 50% | 1 | 5 | Yes | Yes |
| | 650 | | Plus | | | | | |
| Comp. 9^{e} | Silicone | 100% | — | — | 5 | 4 | Yes | No |
| | SSA | | | | | | | |
| Comp. 10^{f} | Acrylic | 100% | — | — | 1 | 5 | Yes | No |
| | Adhesive | | | | | | | |

*Example was the same as Example 6 except no solvent was used. The formulation was blended at 100° C. until homogeneous and then cast/coated from the molten polymer.
^{e}Silicone soft skin adhesive MG7-9900 commercially available from DuPont
^{f}Acrylic pressure sensitive adhesive DURO-TAK ® 387-2510 commercially available from Henkel

### Examples 24-38

Examples 24-38 and Comparative Example 11 below illustrate the effect of polysiloxane concentration on the properties of films (Table 5). Samples were prepared using the preparation techniques described previously. These examples demonstrate that film tack can be adjusted by decreasing the concentration of polysiloxane fluid in the formulations.

TABLE 5
Film formulations and their properties as a function of silicone concentration

| Example | Fluid wt. % | | Polymer | wt. % | Release | Tack | Flexible | Disintegrable |
|---|---|---|---|---|---|---|---|---|
| 24 | Dowsil ® | 62% | Luviskol | 38% | 5 | 4 | Yes | Yes |
| | 4-7042 | | Plus | | | | | |
| 25 | Dowsil | 60% | Luviskol | 40% | 5 | 1 | Yes | Yes |
| | 4-7042 | | Plus | | | | | |
| 26 | Dowsil | 50% | Luviskol | 50% | 5 | 1 | Yes | Yes |
| | 4-7042 | | Plus | | | | | |
| 27 | Dowsil | 62% | Luviskol | 38% | 5 | 4.5 | Yes | Yes |
| | 4-2737 | | Plus | | | | | |
| 28 | Dowsil | 60% | Luviskol | 40% | 5 | 2 | Yes | Yes |
| | 4-2737 | | Plus | | | | | |
| 29 | Dowsil | 50% | Luviskol | 50% | 5 | 1 | Yes | Yes |
| | 4-2737 | | Plus | | | | | |
| 30 | DMS-C16 | 62% | Luviskol | 38% | 4 | 4 | Yes | Yes |
| | | | Plus | | | | | |
| 31 | DMS-C16 | 55% | Luviskol | 45% | 5 | 1 | Yes | Yes |
| | | | Plus | | | | | |
| 32 | DMS-C16 | 50% | Luviskol | 50% | 5 | 1 | Yes | Yes |
| | | | Plus | | | | | |
| 33 | Silmer ® | 68% | Luviskol | 32% | 4 | 4 | Yes | Yes |
| | OHT Di-10 | | Plus | | | | | |
| 34 | Silmer OHT | 62% | Luviskol | 38% | 5 | 3 | Yes | Yes |
| | Di-10 | | Plus | | | | | |
| 35 | Silmer OHT | 60% | Luviskol | 40% | 5 | 1 | Yes | Yes |
| | Di-10 | | Plus | | | | | |
| 36 | Silmer OHT | 50% | Luviskol | 50% | 5 | 1 | Yes | Yes |
| | Di-10 | | Plus | | | | | |
| 37 | Dimethicone | 50% | Luviskol | 50% | 3 | 4 | Yes | Yes |
| | PEG-8 | | Plus | | | | | |
| 38 | Dimethicone | 40% | Luviskol | 60% | 5 | 2 | Yes | Yes |
| | PEG-8 | | Plus | | | | | |
| Comp. 11 | None | 0% | Luviskol | 100% | 5 | 1 | No | Yes |
| | | | Plus | | | | | |

### Examples 39-44

Examples 39-44 show the properties of films made from compositions comprising polymer blends and copolymers (Table 6). Samples were prepared utilizing the preparation techniques described previously.

TABLE 6
Film formulations comprised of polymer blends or copolymers and their properties

| Example | Fluid wt. % | | Polymer | wt. % | Release | Tack | Cohesive | Disintegrable |
|---|---|---|---|---|---|---|---|---|
| 39 | Dowsil ® | 67% | Kollidon | 33% | 5 | 4 | Yes | Yes |
| | 4-7042 | | K90 | | | | | |
| | | | Luviskol | | | | | |
| | | | Plus:50:50 | | | | | |
| 40 | Dowsil | 58% | Kollidon | 42% | 5 | 4 | Yes | Yes |
| | 4-7042 | | K90:Aquazol | | | | | |
| | | | 500 | | | | | |
| | | | 50:50 | | | | | |
| 41 | Dowsil | 66% | Gaffix | 34% | 3 | 3 | Yes | Yes |
| | 4-2737 | | VC-713 | | | | | |
| 42 | Silmer ® | 67% | Gaffix | 33% | 4 | 4 | Yes | Yes |
| | OH Di-10 | | VC-713 | | | | | |
| 43 | Dowsil | 75% | PVP/VA | 25% | 5 | 2 | Yes | Yes |
| | 4-2737 | | E-735 | | | | | |
| 44 | Silmer | 75% | PVP/VA | 25% | 4 | 4 | Yes | Yes |
| | OHT | | E-735 | | | | | |
| | Di-10 | | | | | | | |

### Examples 45-48

Medical adhesive strips and patches were prepared from the formulations described in Examples 3 and 6 as well as Comparative Examples 1 and 9. Solutions of the material were prepared utilizing previously described mixing procedures. The solutions were coated onto 50 μm thick polyester (Mylar) sheets utilizing a Myer rod and 305 μm (12 mil) thick shims. The wet coatings were heat treated in an oven at 130° C. for 4 min. Diamond embossed, low density polyethylene (LDPE) release liner was applied to the surface of the dried coatings utilizing a 15 kg roller. Samples were cut into 2.5 cm strips and allowed to rest for 24 hours under ambient conditions. Evaluation of release and adhesion force in N (newton) for adhesion and release testing were conducted utilizing a Texture Analyzer Model TA-XT Plus with TA-243 attachment. Release testing was accomplished at an angle of 180°, distance of 130 mm and a speed of 10 mm/s to measure the force required to remove the release liner from the adhesive. Afterwards, the adhesive strips were affixed to polycarbonate (PC) and pressed with a 5 kg roller and allowed to dwell for 30 min in preparation for adhesion testing. Adhesion testing to PC was carried out utilizing the same test setup employed for release testing. Samples were tested in triplicate and the average force (N) was reported.

TABLE 7
Release and adhesion force of medical adhesive
strips from LDPE and PC, respectively

| | | Release | Adhesion |
|---|---|---|---|
| Example | Formulation | (N/2.5 cm) | (N/2.5 cm) |
| 45 | Example 3 | 0.1 | 14.6 |
| 46 | Example 6 | 0.4 | 9.2 |
| 47 | Comp. Ex. 1 | Failure | Failure |
| 48 | Comp. Ex. 9 | 0.1 | 4.5 |

### Examples 49-55

Medical electrodes were prepared by incorporating organic salts and electrically conductive additives into the formulation described in Example 6 at various concentrations (Table 8). Benzyltributylammonium bromide and acetylene black were obtained from Sigma Aldrich. Single walled carbon nanotubes were obtained as Tuball 201 from OCSiAl. Formulations with the additives were prepared utilizing mixing procedures described for solution preparation. The solutions were coated onto aluminum foil utilizing a Myer rod and 2 mil thick shims. The wet coatings were heat treated in an oven at 130° C.for 4 min. Circular electrodes were punched from the coated aluminum sheet utilizing a 3.5 cm diameter steel dies. Specimens for electrical performance evaluation were created by placing the electrode adhesive side down onto a second aluminum sheet to form a sandwich configuration. Electrical impedance was measured in a single electrode configuration utilizing a setup according to ANSI/AAMI EC12-2000. A GAG-810 audio generator and B&K 2540B-GEN oscilloscope were employed to measure the electrodes at a setting of 10 Hz and 100 pA.

Patches and strips were also created from the formulations to determine the adhesion and release force. Preparation and testing were consistent with those employed for examples described previously.

TABLE 8
Additives and formulations utilized for creation of medical electrodes.

| | | | wt. % | Coating |
|---|---|---|---|---|
| Example | Formulation | Additive | adhesive | Appearance |
| 49 | Example 6 | None - Control | 0% | Transparent |
| 50 | Example 6 | Benzyltributylammonium | 5% | Transparent |
| | | Bromide | | |
| 51 | Example 6 | Benzyltributylammonium | 10% | Transparent |
| | | Bromide | | |
| 52 | Example 6 | Benzyltributylammonium | 15% | Tinted White |
| | | Bromide | | |
| 53 | Example 6 | Benzyltributylammonium | 20% | Tinted White |
| | | Bromide | | |
| 54 | Example 6 | Acetylene Black | 20% | Black Paste, |
| | | | | Chunks |
| 55 | Example 6 | Single Walled Carbon | 1% | Tinted Black, |
| | | Nanotubes | | Clumps |

TABLE 9
Electrical impedance of electrodes. Adhesion and release force
of medical adhesive strips from PC and LDPE, respectively.

| | Thickness | Impedance | Adhesion | Release |
|---|---|---|---|---|
| Example | μm | kΩ | N/2.5 cm | N/2.5 cm |
| 49 | 58 | 9400 | 9.2 | 0.4 |
| 50 | 99 | 279 | 9.4 | 0.5 |
| 51 | 94 | 288 | 9.3 | 0.7 |
| 52 | 94 | 288 | 8.5 | 0.7 |
| 53 | 99 | 282 | 7.7 | 0.8 |
| 54 | 147 | 37 | 0.0 | 0.0 |
| 55 | 147 | 190 | 3.2 | 0.0 |

### Example 56

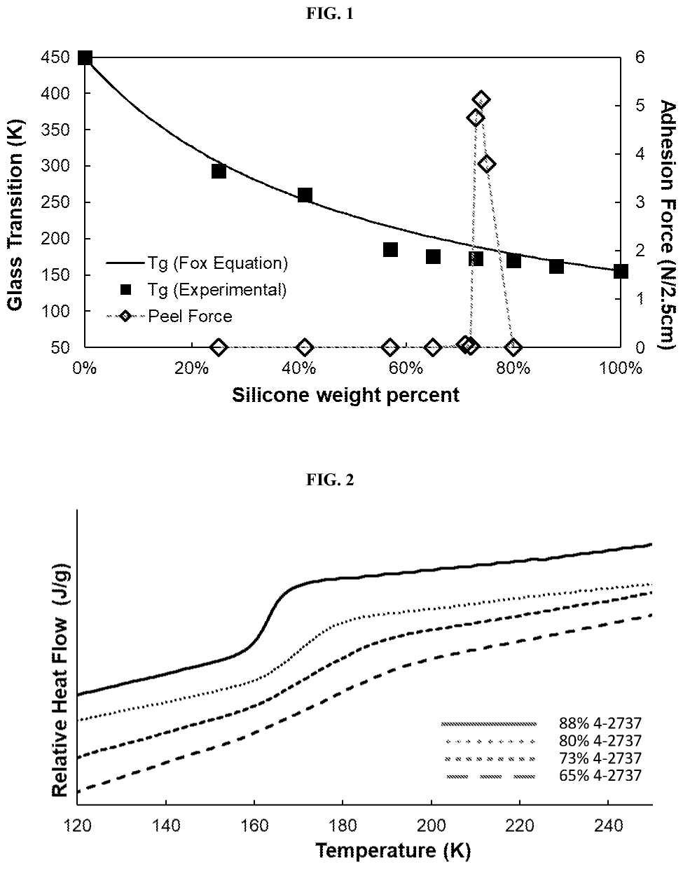

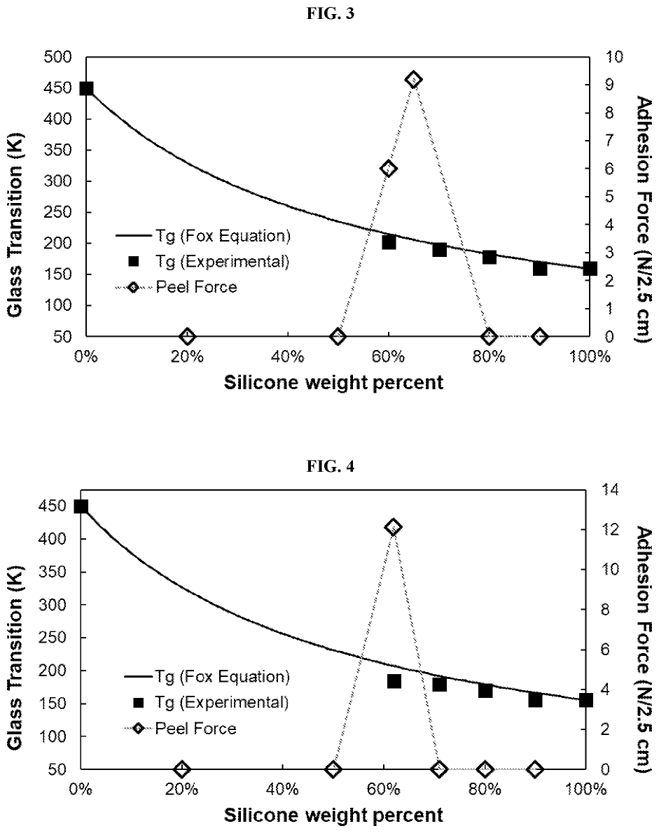

The glass transition temperatures of film-forming compositions were measured and compared against the Fox equation. Blends comprised of polyvinylpyrrolidone and Dowsil® 4-2737 were evaluated and the results are shown in FIG. 1. The measured glass transition temperatures were similar to those predicted by the Fox equation. Some slight deviations between the experimental and predicted glass transition temperatures were observed. These deviations can be attributed to the hydrogen bond interactions between the polymer and the silicone fluid which affected the cooperativity of the blend. Similar deviations in glass transition temperature due to hydrogen bond interactions have been observed in the literature. The peel force of the film-forming compositions from polycarbonate (adhesion force from polycarbonate was evaluated in the same approach described in examples 45-48) indicate that there is a narrow range of compositions which exhibit pressure sensitive adhesion. FIG. 2 shows the differential scanning calorimetry heat flow curves obtained from measuring samples from FIG. 1. A single glass transition temperature was observed for these samples, indicating that homogeneous blends were achieved. FIGS. 3 and 4 contain glass transition temperature and adhesion results from other embodiments of the invention for blends of polycaprolactone and Dowsil® 4-7042 and polyvinylpyrrolidone and Dowsil 4-7042, respectively. These figures illustrate similarly that homogeneous blends were achieved, and that adhesion is dependent upon the blend composition. The films in FIG. 4 had the solvent removed from the mixture before measurement. The highest peel force sample in FIG. 4 corresponds to EXAMPLE 5.

One or more of the values described above may vary by ±5%, ±10%, ±15%, ±20%, ±25%, etc. so long as the variance remains within the scope of the disclosure. Unexpected results may be obtained from each member of a Markush group independent from all other members. Each member may be relied upon individually and or in combination and provides adequate support for specific embodiments within the scope of the appended claims. The subject matter of all combinations of independent and dependent claims, both singly and multiply dependent, is herein expressly contemplated. The disclosure is illustrative including words of description rather than of limitation. Many modifications and variations of the present disclosure are possible in light of the above teachings, and the disclosure may be practiced otherwise than as specifically described herein.

1. A film-forming composition, comprising:
a blend of
i) a polysiloxane comprising one or more hydroxyl groups;
ii) a polymer comprising a nitrogen-containing repeating unit, wherein the nitrogen-containing repeating unit is an amide, tertiary amine, benzylic amine, or heterocyclic amine;
iii) optionally, a solvent;
wherein the film-forming composition forms a film that is water-disintegrable.

2. A film-forming composition according to claim 1, wherein the composition comprises from 35 to 85% (w/w) of i), based on the weight of i) and ii), the hydroxyl content of i) is from 1.5 to 12.0% (w/w), based on the weight of i), and/or the blend is homogeneous.

3. A film-forming composition according to claim 1, wherein the composition comprises from 15% (w/w) to 65% (w/w) of ii), wherein the composition further comprises more than 35% (w/w) of a water-soluble plasticizer, wherein the film-forming composition forms films on skin that once formed only require water to remove from the skin wherein the one or more hydroxyl groups are part of a silanol, polyether, hydroxy-substituted hydrocarbyl, or a combination of two or more of a silanol, polyether, and hydroxyl-substituted hydrocarbyl group and/or wherein the polysiloxane is hydroxy-terminated polydiorganosiloxane; a hydroxyl-substituted hydrocarbyl-terminated polydiorganosiloxane; or a polydiorganosiloxane-polyether copolymer.

4. A film-forming composition according to claim 1, wherein the nitrogen atom of the nitrogen-containing repeating unit is resonance stabilized, or wherein the polymer comprising a nitrogen-containing repeating unit is a homopolymer and copolymer of one or more of vinylcaprolactam, vinylpyrrolidone, 2-methyl-2-oxazoline, or vinylpyridine.

5. A film-forming composition according to claim 1, wherein the composition comprises a solvent, and wherein the solvent is an organic alcohol.

6. A film formed from the composition according to claim 1.

7. A film composition, comprising:
i) a polysiloxane comprising one or more hydroxyl groups; and
ii) a polymer comprising a nitrogen-containing repeating unit, wherein the nitrogen-containing repeating unit is an amide, tertiary amine, benzylic amine, or heterocyclic amine;
wherein the film is water-disintegrable.

8. The film according to claim 7, wherein the film comprises from 35% (w/w) to 85% (w/w) of i), wherein i) comprises from 1.5% (w/w) to 12% (w/w/), based on the weight of i), hydroxyl, and 15% (w/w) to 65% (w/w) of ii).

9. The film according to claim 8, wherein the film will adhere to human skin.

10. The film according to claim 6, wherein the film is a pressure sensitive adhesive.

11. A wearable product, comprising: the film-forming composition according to claim 1, wherein the wearable product is a garment, drape, wound dressing, bandage, patch or medical device.

12. A wearable product, comprising: the film according to claim 7, wherein the wearable product is a medical device and the medical device is a sensor, prosthetic, monitor or defibrillator.

13. A method of protecting skin, the method comprising:
applying the film-forming composition of claim 1 to skin,
wherein the film-forming composition is applied directly to dry skin without pre-treatment with water or a high moisture environment.

14. The method of protecting skin according to claim 13, further comprising applying water to the film, wherein the water reduces the adhesion of the film-forming composition to the skin; and removing the composition from the skin.

15. A method of making the film-forming composition according to any claim 1, comprising:
combining the polysiloxane comprising one or more hydroxyl groups, the polymer comprising a nitrogen-containing repeating unit, wherein the nitrogen-containing repeating unit is an amide, tertiary amine, benzylic amine, or heterocyclic amine, and optionally, the solvent; and
mixing until the blend is formed.
